(19) United States
(12) Patent Application Publication
(10) Pub. No.: US 2012/0302899 A1
(43) Pub. Date: Nov. 29, 2012

(54) SENSOR AND SENSING METHOD THEREOF

(75) Inventors: Jong Gwan YOOK, Seoul (KR); Sanggyu KIM, Seoul (KR); Gi-ho YUN, Gyeonggi-do (KR)

(73) Assignee: Industry-Academic Cooperation Foundation, Yonsei University, Seoul (KR)

(21) Appl. No.: 13/477,152

(22) Filed: May 22, 2012

(30) Foreign Application Priority Data
May 25, 2011 (KR) 10-2011-0049587

(51) Int. Cl. A61B 5/0205

(52) U.S. Cl. 600/484

(57) ABSTRACT

A biosignal measuring sensor and a biosignal measuring method are provided. The biosignal measuring sensor includes an oscillator, a signal detection unit, and a signal processing unit. The amount of oscillation frequency shift of the oscillator is detected as intensity change of a biosignal, and a pulse signal and a respiration signal are detected from the detected biosignal. In the biosignal measuring sensor and the biosignal measuring method, a biosignal is detected in a manner of non-contact with a biosignal measuring target. Moreover, the biosignal may be detected accurately at a close range.

### CROSS-REFERENCE TO RELATED APPLICATIONS

This US non-provisional patent application claims priority under 35 USC §119 to Korean Patent Application No. 10-2011-0049587, filed on May 25, 2011, the entirety of which is hereby incorporated by reference.

### BACKGROUND

The exemplary embodiments of the invention concepts disclosed herein relate to a sensor and a sensing method thereof and, more particularly, to a distance measuring sensor, a biosignal measuring signal, and a biosignal measuring method.

Recently increasing interest in health leads to the rising necessity of home health care services that make it unnecessary to visit special agencies such as hospital. Accordingly, many studies have been conducted to develop biosignal measuring apparatuses that do not have time and space constraints. In addition, with the increase in number of heart-disease deaths and rapid aging of population structure, there is an increasing need for real-time monitoring of biosignals such as a respiration signal and a pulse signal.

In particular, as a demand for medical, military, and automotive applications of non-contact biosignal sensors using RF technologies increases, studies of non-contact biosignal sensors using Doppler Effect have been developed over the past decade. Various hardware systems and software have been proposed to improve performance of such non-contact biosignal sensors using Doppler Effect, which results in the conclusion that a biosignal can be detected at a measurement distance of several meters.

### SUMMARY

Embodiments of the inventive concept provide a biosignal measuring sensor, a terminal, a biosignal measuring method, and a distance measuring sensor.

According to an aspect of the inventive concept, the biosignal measuring sensor may include an oscillator configured to generate oscillation frequency shift in response to a biosignal; and a signal detection unit configured to detect intensity change of the biosignal by using the degree of the oscillation frequency shift.

In an example embodiment, the signal detection unit may include a filter unit configured to detect the intensity change of the biosignal with respect to a frequency by using an SAW filter.

In an example embodiment, the signal detection unit may further include a power detector connected to the filter unit and configured to convert the intensity change of the biosignal with respect to a frequency into the intensity change of the biosignal with respect to time.

In an example embodiment, the oscillator may include a resonator and an input impedance of the resonator may vary with the biosignal. The resonator may be a planar resonator configured to generate resonance at a band from 2.4 GHz to 2.5 GHz.

In an example embodiment, the biosignal measuring sensor may further include a signal processing unit connected to the signal detection unit and configured to detect a pulse signal and a respiration signal from the detected biosignal.

In an example embodiment, the signal processing unit may include a DFT unit configured to transform the detected biosignal in a manner of discrete Fourier transform; a filter unit configured to filter an output signal of the DFT unit to detect a pulse signal and a respiration signal; and an IDFT unit configured to transform the pulse signal and the respiration signal in a manner of inverse discrete Fourier transform.

In an example embodiment, the biosignal measuring sensor may further include a display unit configured to output at least one of the detected pulse and respiration signals.

In an example embodiment, the biosignal measuring sensor may further include an analog-to-digital converter configured to convert the detected biosignal of the intensity change with respect to time into a digital signal; and a data transmitting unit configured to transmit the digital signal to a terminal.

In an example embodiment, the data transmitting unit may be configured to transmit data through a Bluetooth, Wi-Fi or ZigBee communication network.

According to another aspect of the inventive concept, the terminal may include a data receiving unit configured to receive a detected biosignal using oscillation frequency shift of an oscillator corresponding to a biosignal; a signal processing unit configured to detect a pulse signal and a respiration signal from the received biosignal; and a display unit configured to output at least one of the pulse and respiration signals.

According to another aspect of the inventive concept, the biosignal measuring method may include generating oscillation frequency shift of an oscillation corresponding to a biosignal; and detecting intensity change of the biosignal using the degree of the oscillation frequency shift. Generating oscillation frequency shift may include generating oscillation frequency shift by change in input impedance of a resonance.

In an example embodiment, generating oscillation frequency shift may include converting the degree of the oscillation frequency shift into intensity change of the biosignal depending on a frequency; and converting the intensity change of the biosignal depending on frequency into intensity change of the biosignal depending on time.

In an example embodiment, converting the degree of the oscillation frequency shift into intensity change of the biosignal depending on a frequency is performed using an SAW filter.

In an example embodiment, the biosignal measuring method may further include detecting a pulse signal and a respiration signal from the intensity change of the detected biosignal.

In an example embodiment, the biosignal measuring method may further include outputting at least one of the detected pulse and respiration signals.

In the biosignal measuring sensor and the biosignal measuring method, a biosignal is measured in a non-contact manner.

In an example embodiment, the biosignal measuring method may further include transmitting the biosignal detected as the intensity change depending on time to a terminal.

According to another aspect of the inventive concept, the distance measuring sensor may include an oscillator configured to generate oscillation frequency shift corresponding to a measuring target; a storage unit configured to store a shifted oscillation frequency value; and a distance calculator configured to determine a distance to the measuring target based on the shifted oscillation frequency value.

In an example embodiment, the distance measuring sensor may further include a database unit configured to store an oscillation frequency value of the oscillator according to the distance between the measuring target and the distance measuring sensor.

### BRIEF DESCRIPTION OF THE DRAWINGS

The inventive concept will become more apparent in view of the attached drawings and accompanying detailed description. The embodiments depicted therein are provided by way of example, not by way of limitation. The drawings are not necessarily to scale, emphasis instead being placed upon illustrating aspects of the inventive concept.

FIG. 1 is a flowchart illustrating a biosignal measuring method according to one embodiment of the inventive concept.

FIG. 2 illustrates a biosignal measuring sensor according to one embodiment of the inventive concept.

FIG. 3 is a circuit diagram of an oscillator of a biosignal measuring sensor according to one embodiment of the inventive concept.

FIG. 4 illustrates the configuration of a planar resonator of a biosignal measuring sensor according to one embodiment of the inventive concept.

FIG. 5 is a graphic diagram illustrating an output of an oscillator of a biosignal measuring sensor according to one embodiment of the inventive concept.

FIG. 6 is a graphic diagram illustrating oscillation frequency shift of an oscillator of a biosignal measuring sensor according to one embodiment of the inventive concept.

FIG. 7 is a graphic diagram illustrating oscillation frequency shift depending on a distance between a biosignal detection target and a planar resonator according to one embodiment of the inventive concept.

FIG. 8 is a graphic diagram illustrating an output of an SAW filter of a biosignal measuring sensor according to one embodiment of the inventive concept.

FIG. 9 is a graphic diagram illustrating a biosignal measuring result as an output of a power detector of a biosignal measuring sensor according to one embodiment of the inventive concept.

FIG. 10 is a graphic diagram illustrating biosignal magnitude change depending on a distance between a biosignal detection target and a planar resonator according to one embodiment of the inventive concept.

FIG. 11 is a graphic diagram illustrating a result of measuring a discretely-Fourier-transformed biosignal according to one embodiment of the inventive concept.

FIG. 12 is a graphic diagram illustrating a pulse signal detected by a biosignal measuring method according to one embodiment of the inventive concept.

FIG. 13 is a graphic diagram of a pulse signal measured using a pressure sensor to be compared with a biosignal measuring result according to one embodiment of the inventive concept.

FIG. 14 is a graphic diagram of a discretely-Fourier-transformed pulse signal measured using a pressure sensor to be compared with a biosignal measuring result according to one embodiment of the inventive concept.

FIG. 15 is a flowchart illustrating operations of a biosignal measuring sensor and a terminal according to one embodiment of the inventive concept.

FIG. 16 illustrates the configurations of a biosignal measuring sensor and a terminal according to one embodiment of the inventive concept.

FIG. 17 illustrates the configuration of a distance measuring sensor according to one embodiment of the inventive concept.

### DETAILED DESCRIPTION

The inventive concept is described more fully hereinafter with reference to the accompanying drawings, in which embodiments of the inventive concept are shown. This inventive concept may, however, be embodied in many different forms and should not be construed as limited to the embodiments set forth herein. Rather, these embodiments are provided so that this disclosure will be thorough and complete, and will fully convey the scope of the inventive concept to those skilled in the art. The terminology used herein is for the purpose of describing particular embodiments only and is not intended to be limiting of the inventive concept.

As used herein, the singular forms “a”, “an” and “the” are intended to include the plural forms as well, unless the context clearly indicates otherwise. The terms used in the specification are for the purpose of describing particular embodiments only and are not intended to be limiting of the invention.

It will be further understood that the terms “comprises” and/or “comprising”, when used in the specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

Terms “unit”, “block”, “module”, and the like may be used to indicate a unit of processing at least one function or operation. For example, such terms unit”, “block”, and “module” may mean software, or a hardware element such as ASIC or FPGA. However, such terms are not limited to software or hardware. The “unit”, “block”, and “module” may be configured to be included within an addressable storage medium or to operate one or more processors. Thus, “unit”, “block”, and “module” may include constituent elements such as software elements, Object-Oriented software elements, class elements, and task elements, processes, functions, attributes, procedures, sub-routines, segments of program code, drivers, firmware, micro code circuit, data, database, data structures, tables, arrays, and variables. Elements and functions provided within the “unit”, “block”, and “module” may be jointed to reduce the number of elements and the “unit”, “block”, and “module”, or may be additionally divided into elements and “unit”, “block”, and “module”.

In the specification, “biosignal” may be a term encompassing “respiration signal” and “pulse signal” by a heat beat and may mean any biological signals that can be detected from any living organisms. The heart beats with a regular pattern while being repeatedly contracted and dilated. The status of health may be checked by measuring such a regular pattern of heartbeat.

One embodiment of the inventive concept provides a biosignal measuring sensor and a biosignal measuring method, in which biosignal magnitude change is detected using the degree of oscillation frequency shift of an oscillator.

A biosignal measuring sensor and a biosignal measuring method according to one embodiment of the inventive concept will now be described with reference to accompanying drawings.

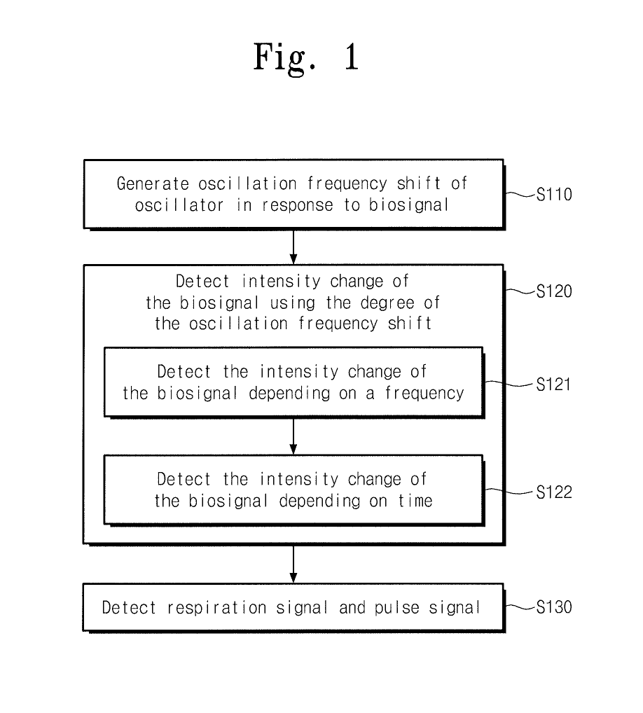

FIG. 1 is a flowchart illustrating a biosignal measuring method according to one embodiment of the inventive concept. As illustrated, the biosignal measuring method may include generating oscillation frequency shift of an oscillator in response to a biosignal (step S110), detecting change in the magnitude of the biosignal using the degree of the oscillation frequency shift (step S120), and detecting a respiration signal and a pulse signal (step S130).

The step S120 may include converting the degree of the oscillation frequency shift into change in the magnitude of the biosignal depending on a frequency (step S121) and converting the change in the magnitude of the biosignal depending on a frequency into change in the magnitude of the biosignal depending on time (step S122).

A biosignal measuring sensor which may be used to perform the method illustrated in FIG. 1 according to one embodiment of the inventive concept will now be described.

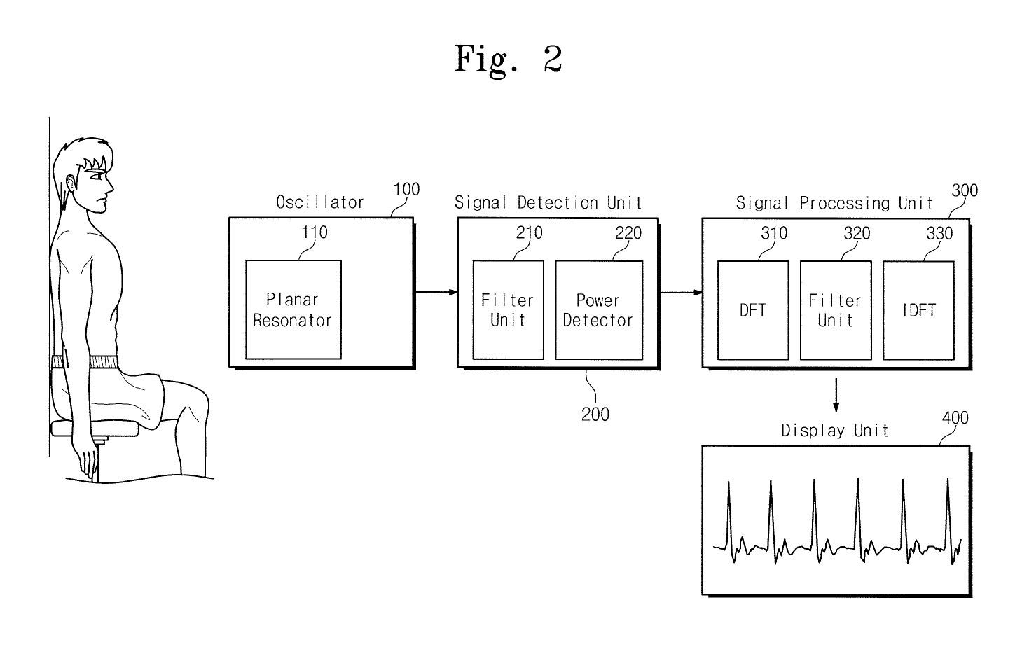

FIG. 2 illustrates a biosignal measuring sensor according to one embodiment of the inventive concept. As illustrated, the biosignal measuring sensor may include an oscillator 100, a signal detection unit 200, a signal processing unit 300, and a display unit 400. The oscillator 100 may include a resonator 110 configured to generate resonance frequency shift. The resonator 110 may include any types of resonators and, preferably, may be a planar resonator. The signal detection unit 200 may include a filter unit 210 configured to convert the degree of the resonance frequency shift into change in the magnitude of a biosignal and a power detector 220 connected to the filter unit 210. The signal processing unit 300 may include a discrete Fourier transform (DFT) unit 310 configured to detect a respiration signal and a pulse signal from the change in the magnitude of a biosignal, a filter unit 320, and an inverse discrete Fourier transform (IDFT) unit 330.

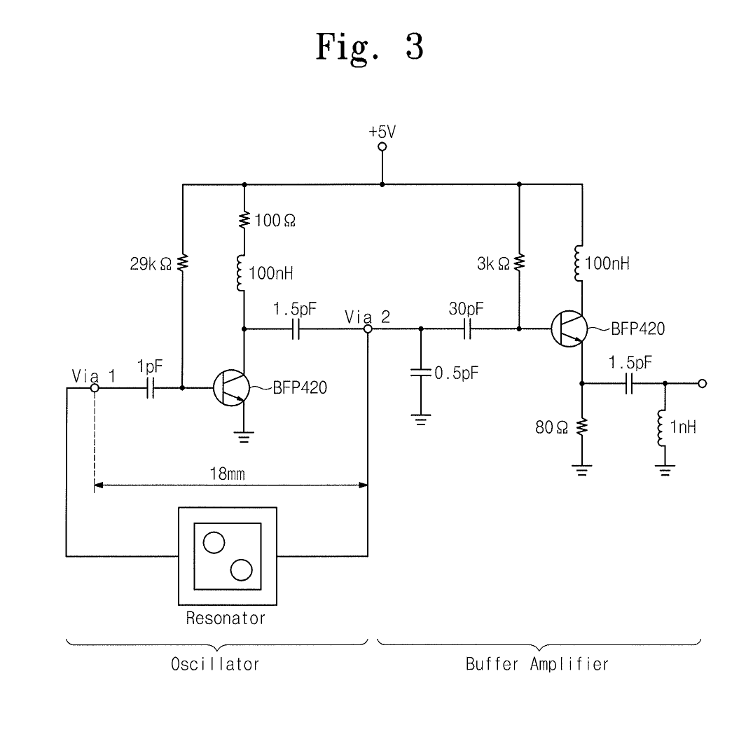

FIG. 3 is a circuit diagram of an oscillator 100 of a biosignal measuring sensor according to one embodiment of the inventive concept. As shown in FIGS. 2 and 3, the oscillator 100 includes a planar resonator 110. A resonator circuit comprises an active circuit unit and a passive circuit unit.

In the oscillator 100, a resistor of 29 KΩ and a resistor of 100Ω may function as a DC bias circuit for setting an operating voltage of a transistor BFP20. An inductor of 100 nH may allow only a DC signal to pass therethrough while an AC signal does not flow to a DC terminal. A capacitor of 1 pF and a capacitor of 1.5 pF may allow a DC voltage to be input only to a transistor. In addition, the capacitor of 1 pF and the capacitor of 1.5 pF may minutely adjust impedance matching between a base and a collector of a transistor and a planar resonator and an oscillation frequency of an oscillator. The planar resonator 110 used as a passive circuit unit may be used as both a sensor and a feedback element satisfying oscillation conditions of an oscillator. The planar resonator 110 is connected to a portion of the base of the transistor in the oscillator. If an impedance of the transistor is adjusted, oscillation conditions of the oscillator may be satisfied at a resonance frequency of the planar resonator. In addition, emitter follower type buffer amplifiers are connected in cascade to prevent malfunction of the oscillator caused by rapid impedance change of the filter unit 210 in the signal detection unit 200 connected to the back end of the oscillator.

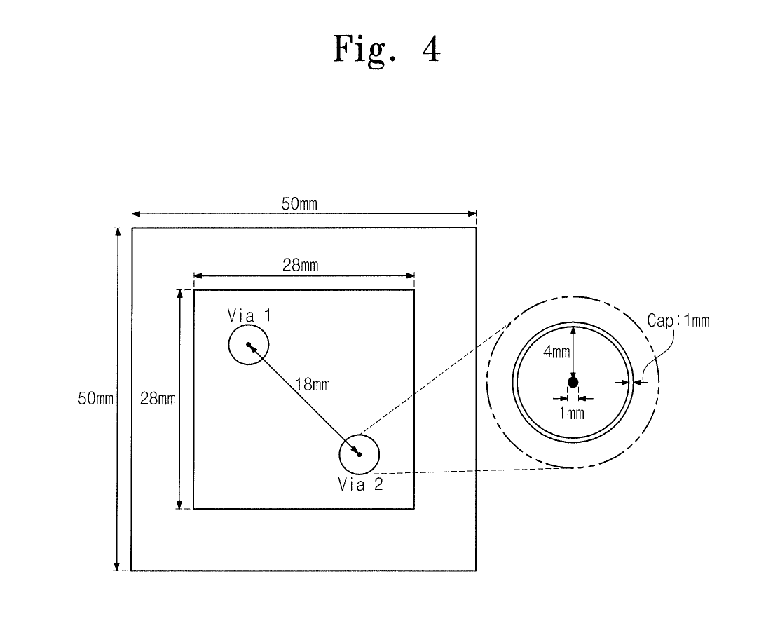

FIG. 4 illustrates the configuration of a planar resonator of a biosignal measuring sensor according to one embodiment of the inventive concept. As illustrated, a planar resonator 110 nay have a size of 28 mm (length)×28 mm (breadth), which is a value determined in the 2.4 GHz industrial scientific medial (ISM) band. The ISM band is a frequency band that can be used in industrial, scientific, and medical applications and includes the band of 2.4 GHz to 2.48 GHz. ISM bands can be used freely (free of charge and without separate frequency authorization from the government). A distance between two terminals of the planar resonator may be about 18 mm in consideration of a distance between a base and a collector of a transistor in an oscillator circuit. A slot of 1 mm may be additionally provided at a portion that is 4 mm from the terminal of the planar resonator in consideration of impedance matching and coupling. A substrate made of an FR4 material (dielectric constant 4.4) may be used and has a size of 50 mm (length)×50 mm (breadth). A result of measuring the planar resonator confirmes that resonance occurs at 2.45 GHz and the planar resonator has 370 3 dB bandwidth of 370 MHz.

When the planar resonator 110 is disposed near (within 20 mm) a biosignal detection target (human body), the periodical movement of the human body caused by respiration generates an additional capacitor element between the planar resonator 110 and the biosignal detection target. The additional capacitor element induces changes of input impedance of the planar resonator 110. The change of input impedance results in oscillation frequency shift of the oscillator 100.

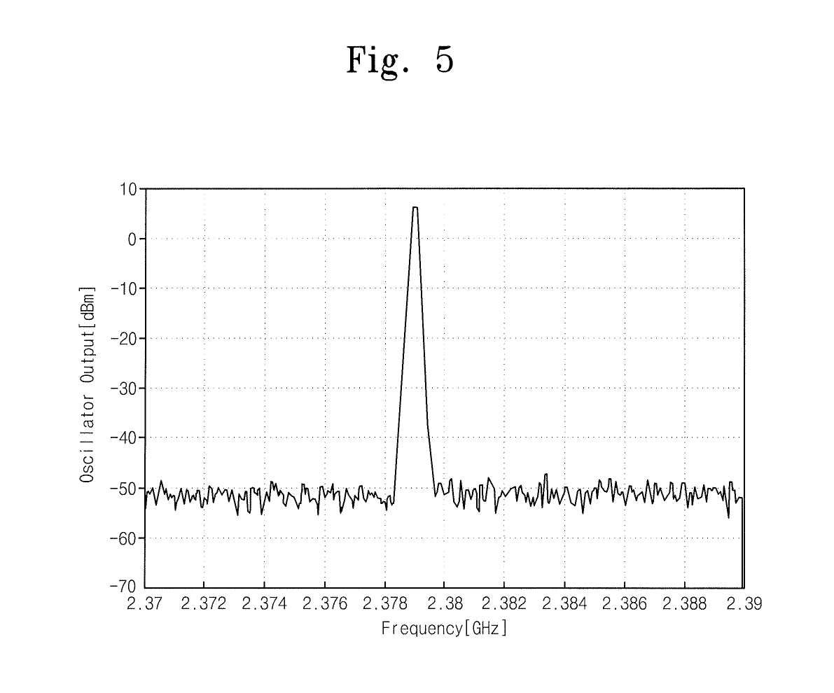

FIG. 5 is a graphic diagram illustrating an output of an oscillator of a biosignal measuring sensor according to one embodiment of the inventive concept. An output of an oscillator was measured by applying a voltage of 5V to an oscillator circuit and using a spectrum analyzer. As illustrated in FIG. 5, the oscillator has a peak output of 4.6 dBm at 2.379 GHz.

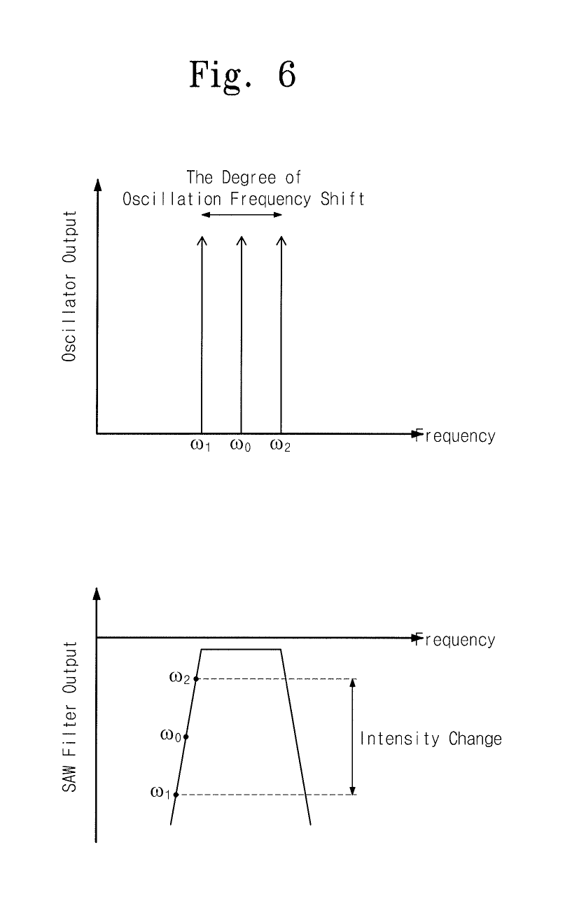

FIG. 6 is a graphic diagram illustrating oscillation frequency shift of an oscillator of a biosignal measuring sensor according to one embodiment of the inventive concept. In the upper graph in FIG. 6, ω₀ represents an oscillation frequency and ω₁ and ω₂ represent shifted oscillation frequencies. For example, if a person inspires in the course of respiration, the oscillation frequency shift occurs in a direction of ω₂. If the person expires in the course of respiration, the oscillation frequency shift occurs in a direction ω₁.

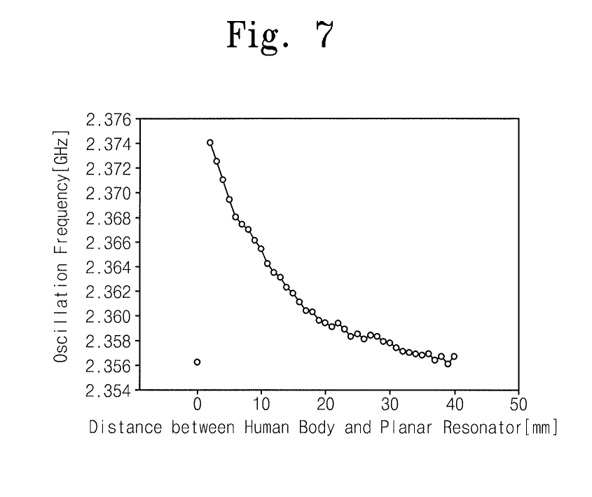

FIG. 7 is a graphic diagram illustrating oscillation frequency shift depending on a distance between a biosignal detection target and a planar resonator according to one embodiment of the inventive concept. When a person did not respire under the state of 0 mm distance between a biosignal detection target and a planar resonator, oscillation frequency shift did not occur and an oscillation frequency of 2.356 GHz was measured. This may imply an initial oscillation frequency when there is no biosignal detection target. In addition, the graph in FIG. 7 may imply that as the distance between a biosignal detection target and a planar resonator increases, the initial frequency when there is no biosignal detection target shifts to an oscillation frequency. When the person respired under the state of 10 mm distance between a biosignal detection target and a planar resonator, oscillation frequency shift of thousands of kHz/mm was measured. When the person respired under the state of 20 mm distance between a biosignal detection target and a planar resonator, oscillation frequency shift of hundreds of kHz/mm was measured.

However, it may be difficult to detect oscillation frequency shift caused by subtle movement (about 1-2 mm) of a biosignal detection target (human body) at a GHz band, as a pulse signal, and convert the oscillation frequency shift into biosignal intensity change. Accordingly, as shown in the lower graph in FIG. 6, it is necessary to convert the degree of oscillation frequency shift into the biosignal intensity change using a filter.

Hereinafter, a procedure in which a signal detection unit 200 converts the degree of oscillation frequency shift into biosignal intensity change will now be described through a filter unit 210 and a power detector 220 that constitute the signal detection unit 200. The filter unit 210 may be connected to an output terminal at the right end of a buffer amplifier of an oscillator circuit.

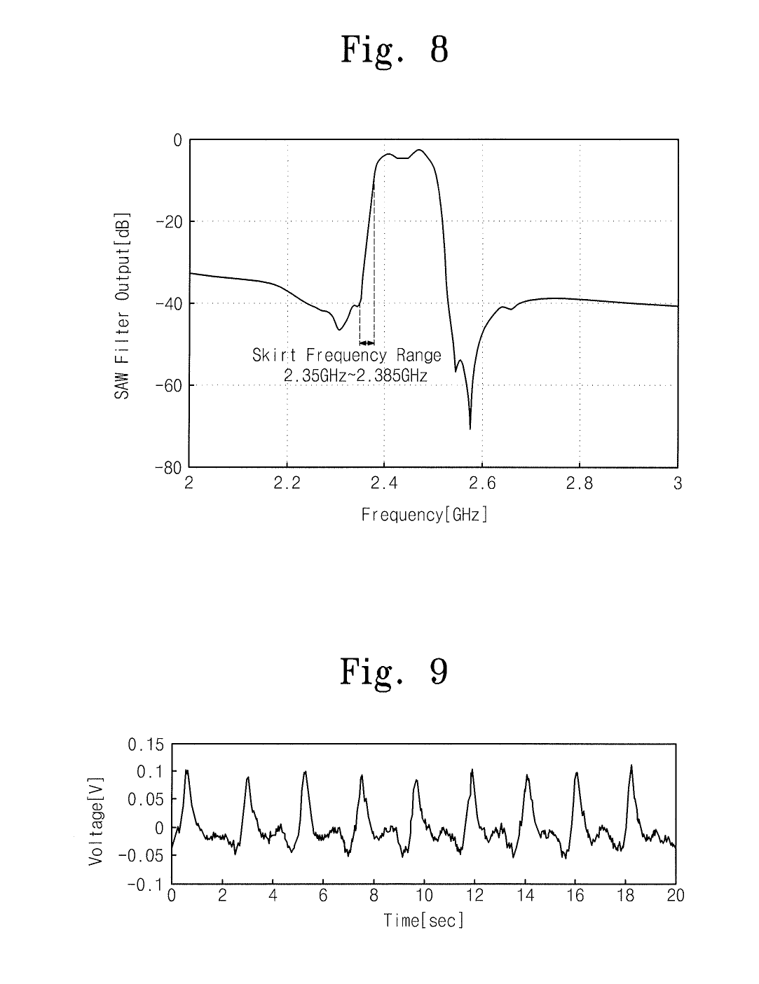

FIG. 8 is a graphic diagram illustrating an output of an SAW filter of a biosignal measuring sensor according to one embodiment of the inventive concept. As set forth above, oscillation frequency shift of about hundreds of kHz/mm occurs when a distance between a planar resonator and a detection target is less than 20 mm. A procedure of converting the degree of the oscillation frequency shift into biosignal intensity change is performed by the filter unit 210 of the signal detection unit 200.

The filter unit 210 may be implemented with a bandpass filter, a highpass filter or a lowpass filter. Preferably, the filter unit 210 may employ a surface acoustic filter. Since the SAW filter has less insertion loss at a pass band while having rapid attenuation characteristics of slope at a low band, the degree of small oscillation frequency shift may be converted into rapid biosignal intensity change. As shown in FIG. 8, the SAW filter according to one embodiment of the inventive concept may detect a biosignal intensity change of 0.1 dB per oscillation frequency shift of 100 kHz in a skirt frequency range from 2.35 GHz to 2.385 GHz. A frequency band of the skirt frequency range is 2.35 GHz to 2.385 GHz, and biosignal intensity change depending on the frequency band of the skirt frequency range is about 35 dB. Accordingly, intensity change of 0.1 dB per oscillation frequency shift of 100 kHz may be calculated. Although a lower frequency band than a pass band of the SAW filter is used, a higher frequency band than the pass band may be used according to an oscillation frequency and the degree of the oscillation frequency shift.

FIG. 9 is a graphic diagram illustrating a biosignal measuring result as an output of a power detector of a biosignal measuring sensor according to one embodiment of the inventive concept. A biosignal intensity change to a frequency detected through the filter unit 210 of the signal detection unit 200 is detected as voltage intensity change of a biosignal depending on time using the power detector 220 connected to the filter unit 210. The power detector 220 may employ an RMS power detector or a peak power detector. The detected voltage intensity change of a biosignal depending on time may be amplified by an operational amplifier. The biosignal intensity change shown in FIG. 9 is the intensity change of a pulse signal whose peak value has the intensity of 0.1 mV. In general, since human body movement caused by respiration (about 5 mm) is much greater than human body movement caused by pulse (about 1-1.5 mm), pulse signal intensity change may not be detected. Accordingly, a signal processing procedure may be required to detect a pulse signal.

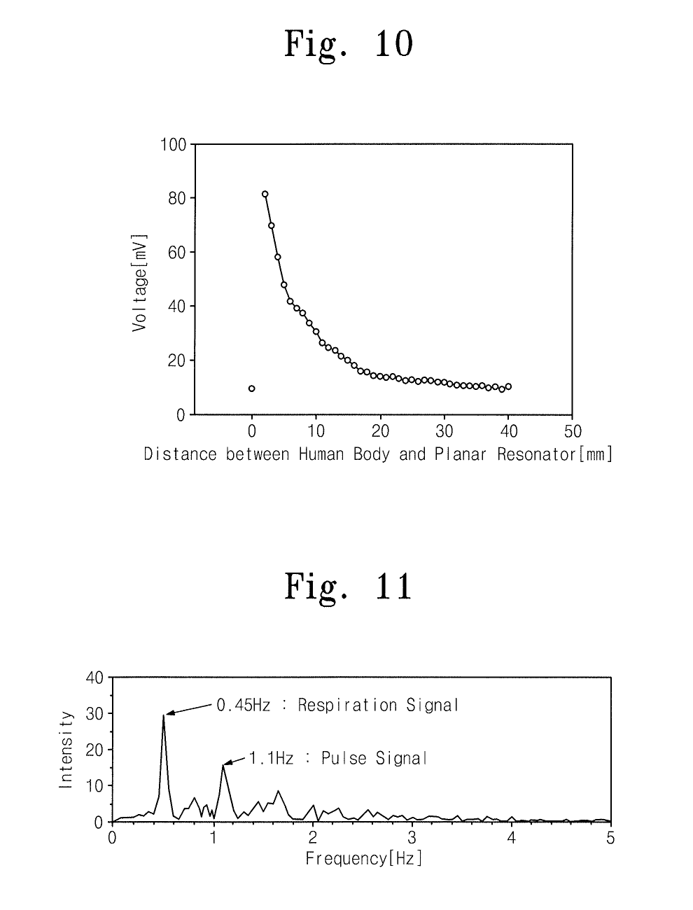

FIG. 10 is a graphic diagram illustrating biosignal magnitude change depending on a distance between a biosignal detection target and a planar resonator according to one embodiment of the inventive concept. As set forth above, the biosignal intensity change of about 0.1 dB per frequency shift of about 100 kHz was detected in the skirt frequency range of 2.35 GHz to 2.385 GHz through the SAW filter. In an interval where a distance between the biosignal detection target and the planar resonator is 10 mm to 20 mm, the biosignal intensity change may be converted into a voltage intensity change of about 1 mV/mm. Thus, the biosignal intensity change may be detected even with respect to subtle movement of a human body such as a pulse. Additionally, in an interval where a distance between the biosignal detection target and the planar resonator is less than 10 mm, the voltage intensity change increases more significantly. Thus, clearer biosignal intensity change may be detected with respect to the subtle movement of a human body such as a pulse signal.

Hereinafter, a procedure in which a signal processing unit 300 detects a pulse signal from detected biosignal intensity change will now be described through a DFT unit 310, a filter unit 320, and an IDFT unit 330. The signal processing unit 300 includes a discrete Fourier transform (DFT) unit 310, a filter unit 320, and an inverse discrete Fourier transform (IDFT) unit 330. The DFT unit 310 transforms biosignal intensity change in a manner of discrete Fourier transform to detect intensity change of a pulse signal.

FIG. 11 is a graphic diagram illustrating a result of measuring a discretely-Fourier-transformed biosignal according to one embodiment of the inventive concept. The discrete Fourier transform may be used in case of obtaining a spectrum sample on frequency from signal samples on time and performed using fast Fourier transform (FFT). In contrast, inverse discrete Fourier transform may be used in case of obtaining a signal sample on time from a spectrum sample on frequency. Referring to FIG. 11, a respiration signal has a peak value at 0.45 Hz and a pulse signal has a peak value at 1.1 Hz. Accordingly, the respiration signal and the pulse signal may be separated using the fact that they have peak values at different frequencies.

The filter unit 320 includes a bandpass filter, a highpass filter, and a lowpass filter. In this embodiment, a pulse signal was filtered using a bandpass filter having a pass band of 5 Hz at 0.8 Hz. However, the pulse signal may be detected using a highpass filter having a pass band of 0.8 Hz or more. In addition, a respiration signal may be detected using a lowpass filter having a pass band of 0.8 Hz or less to be separated from the pulse signal.

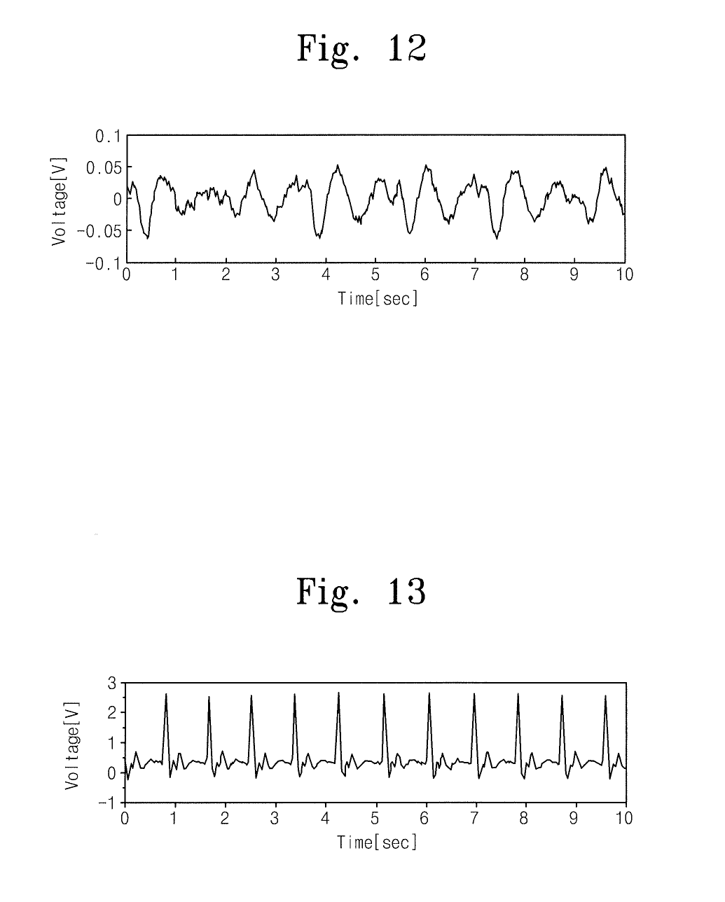

FIG. 12 is a graphic diagram illustrating a pulse signal detected by a biosignal measuring method according to one embodiment of the inventive concept. The pulse signal filtered through the filter unit 320 is input to the IDFT unit 330. The IDFT unit 330 transforms the filtered pulse signal in a manner of inverse discrete Fourier transform to detect pulse signal intensity change depending on time. It will be understood that a peak value of the detected pulse signal is about 0.05 V, which is smaller than that of the respiration signal.

The pulse signal was measured using a finger pressure sensor to be compared with a result of the biosignal measuring sensor and the measuring method according to one embodiment of the inventive concept.

FIG. 13 is a graphic diagram of a pulse signal measured using a pressure sensor to be compared with a biosignal measuring result according to one embodiment of the inventive concept. As compared to FIG. 12, waveforms having peak values are clearly shown. However, it will be confirmed that a graph form is similar to a pulse signal measured using a biosignal measuring sensor according to one embodiment of the inventive concept. This means that as compared to a finger pressure sensor, the biosignal measuring sensor according to one embodiment of the inventive concept may detect the count of the same pulse signal for a fixed measuring time.

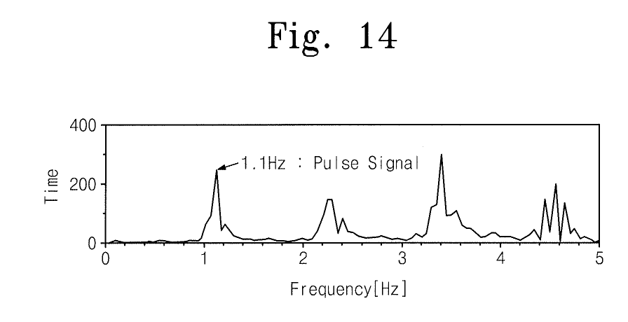

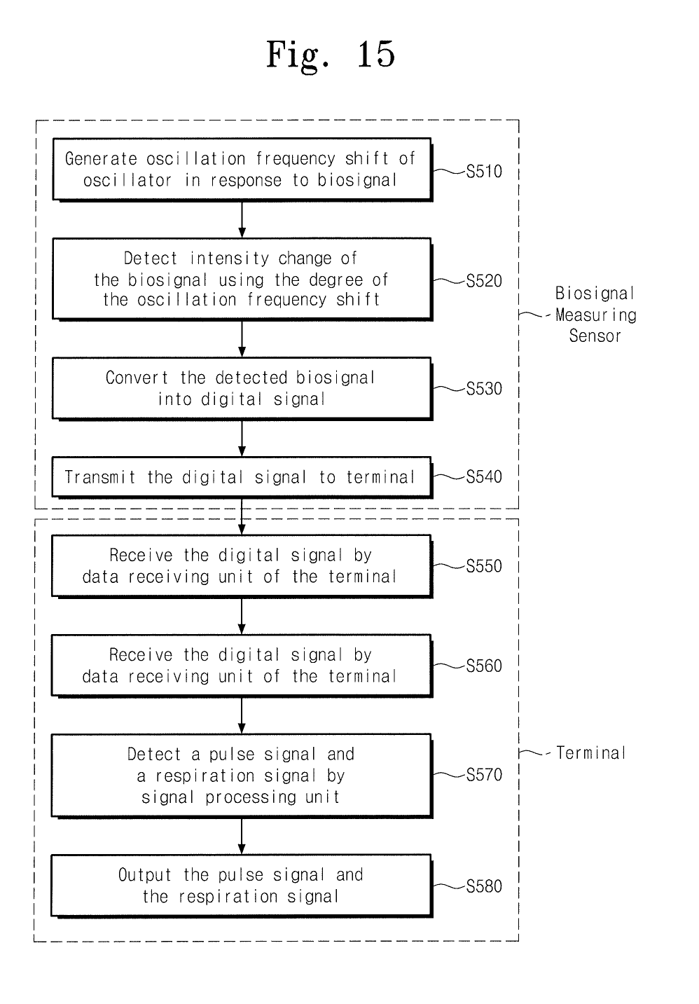

FIG. 14 is a graphic diagram of transforming a pulse signal measured using a finger pressure sensor in a manner of discrete Fourier transform. As shown in FIG. 14, a pulse signal having a peak value at a frequency of 1.1 Hz may be confirmed. It will be confirmed that the pulse signal has a peak value at the same frequency as a pulse signal measured using a biosignal measuring sensor according to one embodiment of the inventive concept.

A biosignal detected through the signal processing unit 300 may be output through a display unit 400. The display unit 400 includes a display panel which visually outputs at least one of detected pulse and respiration signals. The display panel may include a liquid crystal display (LCD), a plasma display panel (PDP), an organic light emitting diode (OLED), and a cathode ray tube (CRT).

A biosignal measuring sensor according to another embodiment of the inventive concept may include a data transfer unit to transfer a detected biosignal to a terminal.

FIG. 15 is a flowchart illustrating operations of a biosignal measuring sensor and a terminal according to another embodiment of the inventive concept. As illustrated in FIG. 15, a biosignal measuring method according to another embodiment of the inventive concept may include generating oscillation frequency shift of an oscillator in response to a biosignal (step S510), detecting intensity change of the biosignal using the degree of the oscillation frequency shift (step S520), converting the detected biosignal into a digital signal (step S530), and transmitting the digital signal to a terminal (step S540).

The terminal according to another embodiment of the inventive concept may operate through the steps that include receiving the digital signal from a biosignal measuring sensor (step S550), converting the received digital signal into an analog signal (step S560), detecting a pulse signal and a respiration signal by a signal processing unit (step S570), and outputting the pulse signal and the respiration signal (step S580).

Now, the configurations of a biosignal measuring sensor and a terminal according to another embodiment of the inventive concept will be described in detail hereinafter to explain the step of the biosignal measuring method and the operation of the terminal.

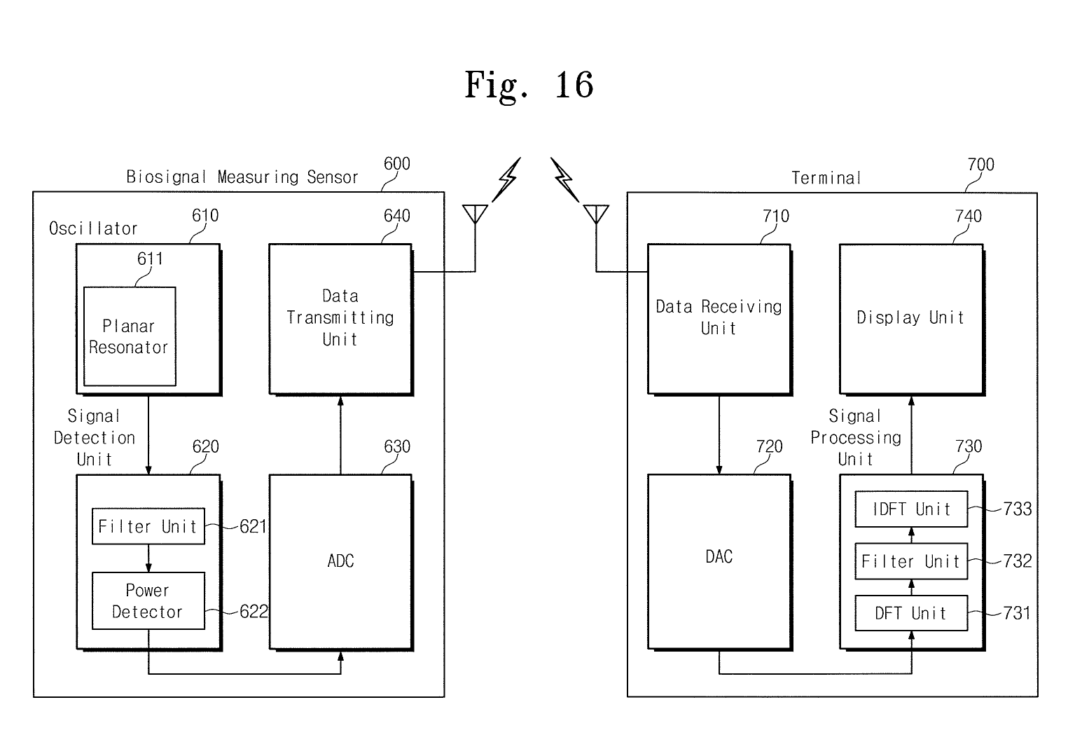

FIG. 16 illustrates the configurations of a biosignal measuring sensor 600 and a terminal 700 according to one embodiment of the inventive concept. As illustrated in FIG. 16, the biosignal measuring sensor 600 may include an oscillator 610, a signal detection unit 620, an analog-to-digital converter (ADC) 630, and a data transmitting unit 640. The oscillator 610 may include a planar resonator 611. The signal detection unit 620 may include a filter unit 621 and a planar resonator 611. The signal detection unit 620 may include a filter unit 621 and a power detector 622. A terminal 700 according to another embodiment of the inventive concept may include a data receiving unit 710, a digital-to-analog converter (DAC) 720, a signal processing unit 730, and a display unit 740. The signal processing unit 730 may include a DFT unit 731, a filter unit 732, and an IDFT unit 733. The detailed explanation of the foregoing configurations will be omitted to avoid duplicate explanation.

The ADC 630 converts an analog signal into a binary digital signal through sampling, quantizing, and binary encoding. The ADC 630 receives an output signal of the power detector 622 and converts the received output signal into a digital signal. The output signal of the power detector 622 may be a biosignal detected as intensity change depending on time. The data transmitting unit 640 may transmit the output signal of the ADC 630 to the terminal 700 through a Bluetooth, Wi-Fi or ZigBee communication network. In addition, the data transmitting unit 640 may communicate with the terminal 700 using a band between 2.3 GHz and 2.5 GHz.

The data receiving unit 710 of the terminal 700 may receive the biosignal converted into the digital signal from the data transmitting unit 640 of the biosignal measuring sensor 600. The received digital signal is input to the DAC 720. The DAC 720 converts the digital signal into an analog signal. The analog signal may be identical to the output signal of the power detector 622 in the signal detection unit 620. The analog signal is input to the signal processing unit 730. The signal processing unit 730 detects a pulse signal and a respiration signal from the analog signal. The DFT unit 731 of the signal processing unit 730 transforms the analog signal in a manner of discrete Fourier transform. The filter unit 732 of the signal processing unit 730 may detect a pulse signal from the discretely-Fourier-transformed signal using a bandpass filter, a highpass filter or a lowpass filter. The IDFT unit 733 of the signal processing unit 730 may transform the detected pulse signal in a manner of inverse discrete Fourier transform into biosignal intensity change depending on time. The display unit 740 of the terminal 700 may include a display panel to visually output at least one of the detected pulse and respiration signals.

As described above, a biosignal measuring sensor and a terminal according to another embodiment of the inventive concept may transmit a detected biosignal to a terminal and output the biosignal through the terminal. Thus, the biosignal may be easily confirmed by a third person such as a guardian beside a biosignal detection target.

According to further another embodiment of the inventive concept, an object (human or thing) distance measuring sensor or an operation detection sensor may be provided. This may be accomplished using a biosignal measuring sensor according to one embodiment of the inventive concept.

Returning to FIG. 7, when there is no object within a measuring distance of the biosignal measuring sensor, the oscillation frequency of the oscillator was measured to be 2.356 GHz. When the distance between an object and the biosignal measuring sensor is 10 mm, the oscillation frequency of the oscillator was measured to be 2.364 GHz. When the distance between an object and the biosignal measuring sensor is 20 mm, the oscillation frequency of the oscillator was measured to be 2.36 GHz. Likewise, since oscillation frequency shift of the oscillator occurs when there is an object within the measuring distance of the sensor, the biosignal measuring sensor according to one embodiment of the inventive concept may be used as an operation detection sensor of the object. For example, the biosignal measuring sensor according to one embodiment of the inventive concept may be used as a movement detection sensor for security.

Now, a distance measuring sensor according to another embodiment of the inventive concept will be described in detail hereinafter.

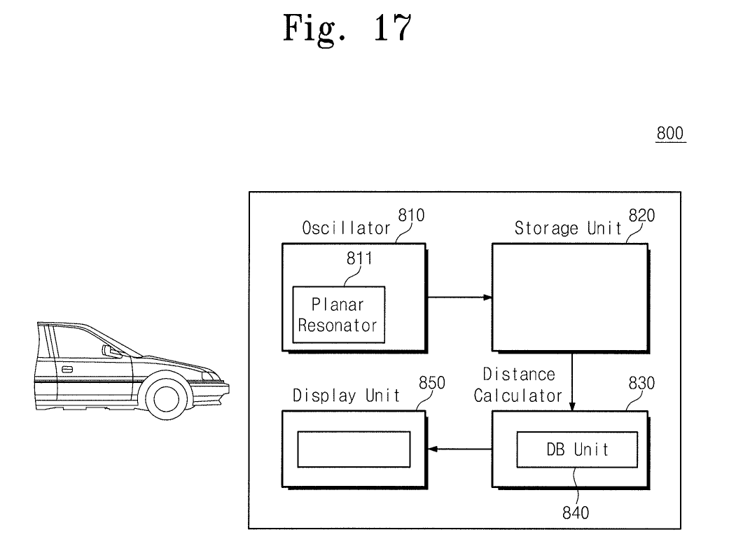

FIG. 17 is a block diagram illustrating the configuration of a distance measuring sensor 800 according to another embodiment of the inventive concept. As illustrated in FIG. 17, the distance measuring sensor 800 may include an oscillator 810 configured to generate oscillation frequency shift of an oscillator corresponding to a measuring target, a storage unit 820 configured to a value of the shifted oscillation frequency, a distance calculator 830 configured to determine a distance to the measuring target based on the value of the shifted oscillation frequency, and a display unit 850 configured to output a distance from the sensor to the measuring target. The distance calculator 830 may further include a database (DB) unit 840 configured to store an oscillation frequency value of the oscillator according to the distance between a measuring target and a position measuring sensor.

For example, when a distance between a vehicle and a sensor is 20 mm, an oscillation frequency of an oscillator is shifted with 2.7 GHz. The oscillation frequency shift is measured and stored in the DB unit 840 after being converted into database. If the vehicle approaches the sensor to cause the oscillation frequency of the oscillator to be shifted with 2.7 GHz, the distance calculator 830 may compare the oscillation frequency shift with an oscillation frequency value stored in the DB unit 840. As a result, it may be determined that the distance between the vehicle and the sensor is 20 mm.

As set forth above, according to further another embodiment of the inventive concept, a distance measuring sensor and an operation detection sensor may be provided.

According to the embodiments of the inventive concept described above, a biosignal of a biosignal detection target can be accurately measured even when there are a number of persons within a measuring distance. In addition, a distance between a measuring target and a sensor can be accurately measured.

While the inventive concept has been particularly shown and described with reference to exemplary embodiments thereof, it will be apparent to those of ordinary skill in the art that various changes in form and detail may be made therein without departing from the spirit and scope of the inventive concept as defined by the following claims.

What is claimed is:

1. A biosignal measuring sensor comprising:
an oscillator configured to generate oscillation frequency shift in response to a biosignal; and
a signal detection unit configured to detect intensity change of the biosignal by using the degree of the oscillation frequency shift.

2. The biosignal measuring sensor of claim 1, wherein the signal detection unit comprises a filter unit configured to detect the intensity change of the biosignal with respect to a frequency by using an SAW filter.

3. The biosignal measuring sensor of claim 2, wherein the signal detection unit further comprises a power detector connected to the filter unit and configured to convert the intensity change of the biosignal with respect to a frequency into the intensity change of the biosignal with respect to time.

4. The biosignal measuring sensor of claim 3, further comprising:
an analog-to-digital converter configured to convert the detected biosignal of the intensity change with respect to time into a digital signal; and
a data transmitting unit configured to transmit the digital signal to a terminal.

5. The biosignal measuring sensor of claim 4, wherein the data transmitting unit is configured to transmit data through a Bluetooth, Wi-Fi or ZigBee communication network.

6. The biosignal measuring sensor of claim 1, wherein the oscillator comprises a resonator, and
wherein an input impedance of the resonator varies with the biosignal.

7. The biosignal measuring sensor of claim 6, wherein the resonator comprises a planar resonator configured to generate resonance at a band from 2.4 GHz to 2.5 GHz.

8. The biosignal measuring sensor of claim 1, further comprising:
a signal processing unit connected to the signal detection unit and configured to detect a pulse signal and a respiration signal from the detected biosignal.

9. The biosignal measuring sensor of claim 8, wherein the signal processing unit comprises:
a DFT unit configured to transform the detected biosignal in a manner of discrete Fourier transform;
a filter unit configured to filter an output signal of the DFT unit to detect a pulse signal and a respiration signal; and
an IDFT unit configured to transform the pulse signal and the respiration signal in a manner of inverse discrete Fourier transform.

10. The biosignal measuring sensor of claim 8, further comprising:
a display unit configured to output at least one of the detected pulse and respiration signals.

11. The biosignal measuring sensor of claim 10, which measures a biosignal in a non-contact manner.

12. A terminal comprising:
a data receiving unit configured to receive a detected biosignal using oscillation frequency shift of an oscillator corresponding to a biosignal;
a signal processing unit configured to detect a pulse signal and a respiration signal from the received biosignal; and
a display unit configured to output at least one of the pulse and respiration signals.

13. A biosignal measuring method comprising:
generating oscillation frequency shift of an oscillation corresponding to a biosignal; and
detecting intensity change of the biosignal using the degree of the oscillation frequency shift.

14. The biosignal measuring method of claim 13, wherein generating oscillation frequency shift comprises:
generating oscillation frequency shift by change in input impedance of a resonance.

15. The biosignal measuring method of claim 13, wherein generating oscillation frequency shift comprises:
converting the degree of the oscillation frequency shift into intensity change of the biosignal depending on a frequency; and
converting the intensity change of the biosignal depending on frequency into intensity change of the biosignal depending on time.

16. The biosignal measuring method of claim 15, further comprising:
transmitting the biosignal detected as the intensity change depending on time to a terminal.

17. The biosignal measuring method of claim 15, wherein converting the degree of the oscillation frequency shift into intensity change of the biosignal depending on a frequency is performed using an SAW filter.

18. The biosignal measuring method of claim 15, wherein converting the intensity change of the biosignal depending on frequency into intensity change of the biosignal depending on time is performed using a power detector.

19. The biosignal measuring method of claim 13, further comprising:
detecting a pulse signal and a respiration signal from the intensity change of the biosignal.

20. A distance measuring sensor comprising:
an oscillator configured to generate oscillation frequency shift corresponding to a measuring target;
a storage unit configured to store a shifted oscillation frequency value; and
a distance calculator configured to determine a distance to the measuring target based on the shifted oscillation frequency value.

21. The distance measuring sensor further comprising:
a database unit configured to store an oscillation frequency value of the oscillator according to the distance between the measuring target and the distance measuring sensor.
